(19) United States
(12) Patent Application Publication
(10) Pub. No.: US 2007/0032468 A1
(43) Pub. Date: Feb. 8, 2007

(54) Novel thioxanthine derivatives for use as inhibitors of mpo

(75) Inventors: Anthony Kettle, Christchurch (NZ); Anna-Karin Tiden, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertaije (SE)

(21) Appl. No.: 10/575,592

(22) PCT Filed: Oct. 14, 2004
(86) PCT No.: PCT/SE04/01477

§ 371 Date: Apr. 12, 2006

(30) Foreign Application Priority Data
Oct. 17, 2003 (SE) 0302756-2

(51) Int. Cl. A61K 31/553; A61K 31/554; A61K 31/551; A61K 31/55; A61K 31/541; A61K 31/5377; A61K 31/522; C07D 473/02

(52) U.S. Cl. 514/210.21; 514/217.06; 514/211.15; 514/218; 514/227.8; 514/234.5; 514/263.34; 514/263.2; 544/60; 544/118; 544/267; 544/269; 540/600; 540/544; 540/575

(57) ABSTRACT

There are disclosed novel compounds of formula (Ia) or (Ib) 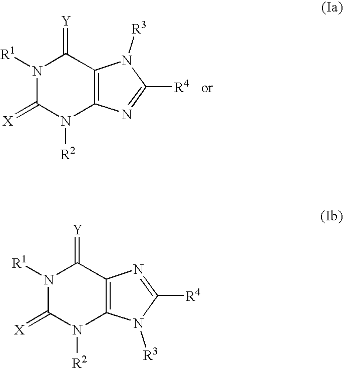
wherein R¹, R², R³, R⁴, X and Y are as defined in the specification, and pharmaceutically acceptable salts thereof; together with processes for their preparation, compositions containing them and their use in therapy. The compounds are inhibitors of the enzyme MPO and are thereby particularly useful in the treatment or prophylaxis of neuroinflammatory disorders.

### FIELD OF THE INVENTION

The present invention relates to novel thioxanthine derivatives, processes for their preparation, compositions containing them and their use in therapy.

### BACKGROUND OF THE INVENTION

Myeloperoxidase (MPO) is a heme-containing enzyme found predominantly in polymorphonuclear leukocytes (PMNs). MPO is one member of a diverse protein family of mammalian peroxidases that also includes eosinophil peroxidase, thyroid peroxidase, salivary peroxidase, lactoperoxidase, prostaglandin H synthase, and others. The mature enzyme is a dimer of identical halves. Each half molecule contains a covalently bound heme that exhibits unusual spectral properties responsible for the characteristic green colour of MPO. Cleavage of the disulphide bridge linking the two halves of MPO yields the hemi-enzyme that exhibits spectral and catalytic properties indistinguishable from those of the intact enzyme. The enzyme uses hydrogen peroxide to oxidize chloride to hypochlorous acid. Other halides and pseudohalides (like thiocyanate) are also physiological substrates to MPO.

PMNs are of particular importance for combating infections. These cells contain MPO, with well documented microbicidal action. PMNs act non-specifically by phagocytosis to engulf microorganisms, incorporate them into vacuoles, termed phagosomes, which fuse with granules containing myeloperoxidase to form phagolysosomes. In phagolysosomes the enzymatic activity of the myeloperoxidase leads to the formation of hypochlorous acid, a potent bactericidal compound. Hypochlorous acid is oxidizing in itself, and reacts most avidly with thiols and thioethers, but also converts amines into chloramines, and chlorinates aromatic amino acids. Macrophages are large phagocytic cells which, like PMNs, are capable of phagocytosing microorganisms. Macrophages can generate hydrogen peroxide and upon activation also produce myeloperoxidase. MPO and hydrogen peroxide can also be released to the outside of the cells where the reaction with chloride can induce damage to adjacent tissue.

Linkage of myeloperoxidase activity to disease has been implicated in neurological diseases with a neuroinflammatory response including multiple sclerosis, Alzheimer's disease, Parkinson's disease and stroke as well as other inflammatory diseases or conditions like asthma, chronic obstructive pulmonary disease, cystic fibrosis, atherosclerosis, inflammatory bowel disease, renal glomerular damage and rheumatoid arthritis. Lung cancer has also been suggested to be associated with high MPO levels.

The present invention discloses novel thioxanthine derivatives that surprisingly display useful properties as inhibitors of the enzyme MPO.

### DISCLOSURE OF THE INVENTION

The present invention provides a compound of formula (Ia) or (Ib)

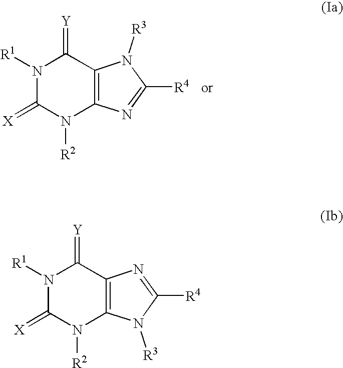

wherein:

one of X and Y represents S, and the other represents O or S;

R¹ represents hydrogen or C1 to 6 alkyl;

R² represents hydrogen or C1 to 6 alkyl; said alkyl group being optionally substituted by:

i) a saturated or partially unsaturated 3- to 7-membered ring optionally incorporating one or two heteroatoms selected independently from O, N and S, and optionally incorporating a carbonyl group; said ring being optionally substituted by one or more substituents selected from halogen, hydroxy, C1 to 6 alkoxy and C1 to 6 alkyl; said alkyl being optionally further substituted by hydroxy or C1 to 6 alkoxy; or

ii) C1 to 6 alkoxy; or

iii) an aromatic ring selected from phenyl, furyl or thienyl; said aromatic ring being optionally further substituted by halogen, C1 to 6 alkyl or C1 to 6 alkoxy;

R³ represents hydrogen or C1 to 6 alkyl;

R⁴ represents halogen, C1 to 6 alkyl substituted by one or more halogen atoms, C1 to 6 alkoxy or C1 to 6 thioalkoxy; said alkoxy or thioalkoxy group being optionally further substituted by halogen or OH;

and pharmaceutically acceptable salts thereof.

The compounds of formula (Ia) or (Ib) may exist in enantiomeric forms. It is to be understood that all enantiomers, diastereomers, racemates and mixtures thereof are included within the scope of the invention.

It will be appreciated that when R³ in formulae (Ia) and (Ib) represents hydrogen, the two alternative representations (Ia) and (Ib) are tautomeric forms of the same compound. All such tautomers and mixtures of tautomers are included within the scope of the present invention.

Unless otherwise indicated, the term “C1 to 6 alkyl” referred to herein denotes a straight or branched chain alkyl group having from 1 to 6 carbon atoms. Examples of such groups include methyl, ethyl, 1-propyl, n-butyl, iso-butyl, tert-butyl, pentyl and hexyl.

The term “C1 to 4 alkyl” is to be interpreted analogously.

Unless otherwise indicated, the term “C3 to 7 cycloalkyl” referred to herein denotes a cyclic alkyl group having from 3 to 7 carbon atoms. Examples of such groups include cyclopropyl, cyclopentyl and cyclohexyl.

Unless otherwise indicated, the term “C1 to 6 alkoxy” referred to herein denotes a straight or branched chain alkoxy group having from 1 to 6 carbon atoms. Examples of such groups include methoxy, ethoxy, 1-propoxy, 2-propoxy and tert-butoxy.

The term “C1 to 4 alkoxy” is to be interpreted analogously.

Unless otherwise indicated, the term “C1 to 6 thioalkoxy” referred to herein denotes a straight or branched chain alkyl group having from 1 to 6 carbon atoms bonded to a sulphur atom. Examples of such groups include methylthio, ethylthio, 1-propylthio, 2-propylthio and tert-butylthio.

Unless otherwise indicated, the term “halogen” referred to herein denotes fluoro, chloro, bromo and iodo.

Examples of a saturated or partially unsaturated 3- to 7-membered ring optionally incorporating one or two heteroatoms selected independently from O, N and S, and optionally incorporating a carbonyl group include cyclopropyl, cyclopentyl, cyclohexyl, cyclopentanone, tetrahydrofuran, pyrrolidine, piperidine, morpholine, piperazine, pyrrolidinone and piperidinone. Particular examples include cyclopropyl, cyclohexyl, tetrahydrofuranyl(tetrahydrofuryl) and morpholinyl.

Examples of a C1 to 6 alkyl substituted by one or more halogen atoms include chloromethyl, 2,2,2-trichloroethyl, fluoromethyl, difluoromethyl, trifluoromethyl, 2,2,2-trifluoroethyl, 1,1-difluoroethyl, pentafluoroethyl and 3,3,3-trifluoropropyl.

In one embodiment, the invention relates to compounds of formula (Ia) or (Ib) wherein X represents S and Y represents O.

In another embodiment, R³ in formula (Ia) or (Ib) represents hydrogen.

In another embodiment, R² in formula (Ia) or (Ib) represents optionally substituted C1 to 6 alkyl.

In another embodiment, R² in formula (Ia) or (Ib) represents C1 to 6 alkyl substituted by a saturated or partially unsaturated 3- to 7-membered ring optionally incorporating one or two heteroatoms selected independently from O, N and S, and optionally incorporating a carbonyl group; said ring being optionally substituted by one or more substituents selected from halogen, hydroxy, C1 to 6 alkoxy and C1 to 6 alkyl; said alkyl being optionally further substituted by hydroxy or C1 to 6 alkoxy.

In another embodiment, R² in formula (Ia) or (Ib) represents methylene, ethylene or trimethylene substituted by cyclopropyl, cyclohexyl, tetrahydrofuranyl or morpholinyl.

In another embodiment, R² in formula (Ia) or (Ib) represents C1 to 6 alkyl substituted by C1 to 6 alkoxy.

In another embodiment, R² in formula (Ia) or (Ib) represents ethylene or trimethylene substituted by methoxy or ethoxy.

In another embodiment, R² in formula (Ia) or (Ib) represents C1 to 6 alkyl substituted by optionally substituted phenyl, furyl or thienyl.

In another embodiment, R⁴ in formula (Ia) or (Ib) represents C1 to 6 alkyl substituted by one or more halogen atoms. In another embodiment, R² in formula (Ia) or (Ib) represents C1 to 6 alkyl substituted by one or more fluoro atoms.

When X represents S and Y represents O, a further embodiment comprises compounds of formula (Ia) or (Ib) wherein R¹ represents hydrogen.

When X represents O and Y represents S, a further embodiment comprises compounds of formula (Ia) or (Ib) wherein R¹ represents C1 to 6 alkyl.

In one embodiment, there are provided compounds of formula (Ia) or (Ib) wherein at least one of X and Y represents S, and the other represents O or S; R¹ represents hydrogen or C1 to 6 alkyl; R² represents hydrogen or C1 to 6 alkyl; said alkyl group being optionally substituted by C3 to 7 cycloalkyl, C1 to 4 alkoxy, or an aromatic ring selected from phenyl, furyl or thienyl; said aromatic ring being optionally further substituted by halogen, C1 to 4 alkyl or C1 to 4 alkoxy; R³ represents hydrogen or C1 to 6 alkyl; and pharmaceutically acceptable salts thereof.

In another embodiment, there are provided compounds of formula (Ia) or (Ib) wherein at least one of X and Y represents S, and the other represents O or S; R¹ represents hydrogen or C1 to 6 alkyl; R² represents hydrogen or C1 to 6 alkyl; said alkyl group being optionally substituted by: i) a saturated or partially unsaturated 3- to 7-membered ring optionally incorporating one or two heteroatoms selected independently from O, N and S, and optionally incorporating a carbonyl group; said ring being optionally substituted by one or more substituents selected from halogen, hydroxy, C1 to 6 alkoxy and C1 to 6 alkyl; said alkyl being optionally further substituted by hydroxy or C1 to 4 alkoxy; or ii) C1 to 4 alkoxy; or iii) an aromatic ring selected from phenyl, furyl or thienyl; said aromatic ring being optionally further substituted by halogen, C1 to 4 alkyl or C1 to 4 alkoxy; R³ represents hydrogen or C1 to 6 alkyl; and pharmaceutically acceptable salt thereof.

In one embodiment, the invention relates to compounds of formula (Ia) or (Ib) wherein X represents S and Y represents O; R² represents optionally substituted C1 to 6 alkyl; and R¹ and R³ each represent hydrogen.

In one embodiment, the invention relates to compounds of formula (Ia) or (Ib) wherein X represents S and Y represents O; R² represents C1 to 6 alkyl substituted by a saturated or partially unsaturated 3- to 7-membered ring optionally incorporating one or two heteroatoms selected independently from O, N and S, and optionally incorporating a carbonyl group; said ring being optionally substituted by one or more substituents selected from halogen, hydroxy, C1 to 6 alkoxy and C1 to 6 alkyl; said alkyl being optionally further substituted by hydroxy or C1 to 6 alkoxy; and R¹ and R³ each represent hydrogen.

In one embodiment, the invention relates to compounds of formula (Ia) or (Ib) wherein X represents S and Y represents O; R² represents C1 to 6 alkyl substituted by C1 to 6 alkoxy; and R¹ and R³ each represent hydrogen.

Particular compounds of the invention include:

3-isobutyl-2-thioxo-8-trifluoromethyl-1,2,3,7-tetrahydro-purin-6-one;

and pharmaceutically acceptable salts thereof.

A further aspect of the invention is the use of the novel compounds of formula (Ia) or (Ib) as a medicament.

A further aspect of the invention is the use of a compound of formula (Ia) or (Ib), or a pharmaceutically acceptable salt thereof, in the manufacture of a medicament, for the treatment or prophylaxis of diseases or conditions in which inhibition of the enzyme MPO is beneficial.

A more particular aspect of the invention provides the use of a compound of formula (Ia) or (Ib), or a pharmaceutically acceptable salt thereof, in the manufacture of a medicament, for the treatment or prophylaxis of neuroinflammatory disorders.

Another more particular aspect of the invention provides the use of a compound of formula (Ia) or (Ib), or a pharmaceutically acceptable salt thereof, in the manufacture of a medicament, for the treatment or prophylaxis of multiple sclerosis.

According to the invention, there is also provided a method of treating, or reducing the risk of, diseases or conditions in which inhibition of the enzyme MPO is beneficial which comprises administering to a person suffering from or at risk of, said disease or condition, a therapeutically effective amount of a compound of formula (Ia) or (Ib), or a pharmaceutically acceptable salt thereof.

More particularly, there is also provided a method of treating, or reducing the risk of, neuroinflammatory disorders in a person suffering from or at risk of, said disease or condition, wherein the method comprises administering to the person a therapeutically effective amount of a compound of formula (Ia) or (Ib), or a pharmaceutically acceptable salt thereof.

In another aspect the invention provides a pharmaceutical formulation comprising a therapeutically effective amount of a compound of formula (Ia) or (Ib), or a pharmaceutically acceptable salt thereof, in admixture with a pharmaceutically acceptable adjuvant, diluent or carrier, for use in the treatment or prophylaxis of diseases or conditions in which inhibition of the enzyme MPO is beneficial.

In another more particular aspect the invention provides a pharmaceutical formulation comprising a therapeutically effective amount of a compound of formula (Ia) or (Ib), or a pharmaceutically acceptable salt thereof, in admixture with a pharmaceutically acceptable adjuvant, diluent or carrier, for use in the treatment or prophylaxis of neuroinflammatory disorders.

According to the invention, we further provide a process for the preparation of the novel compounds of formula (Ia) or (Ib), or a pharmaceutically acceptable salt, enantiomer, diastereomer or racemate thereof which comprises:

(a) reaction of a compound of formula (IIa) or (IIb)

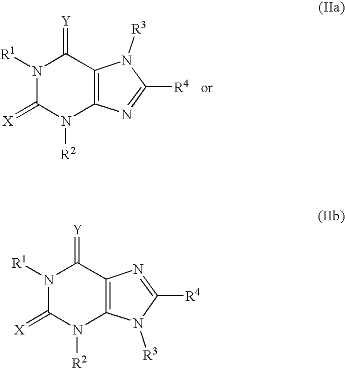

wherein R¹, R², R³ and R⁴ are as defined in formula (Ia) or (Ib), X represents O or S and Y represents O;

with a sulphurising compound such as Lawesson's reagent or phosphorus pentasulphide; to give a corresponding compound wherein Y represents S; or

(b) reaction of a diamine of formula (IIIa) or (IIIb)

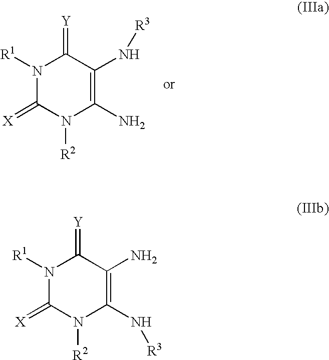

wherein R¹, R², R³, X and Y are as defined in formula (Ia) or (Ib);

with a trialkylorthoester or with an alpha-halo-substituted carboxylic acid or anhydride;

and where necessary converting the resultant compound of formula (Ia) or (Ib), or another salt thereof, into a pharmaceutically acceptable salt thereof; or converting the resultant compound of formula (Ia) or (Ib) into a further compound of formula (Ia) or (Ib); and where desired converting the resultant compound of formula (Ia) or (Ib) into an optical isomer thereof.

In process (a), a compound of formula (IIa) or (IIb) and a sulfurising agent such as Lawesson's reagent, or phosphorus pentasulfide are dissolved or suspended in a suitable dry organic solvent such as benzene, toluene, xylene, tetrahydrofuran, dichloromethane or dioxane and then heated to between 30° C. and the reflux temperature of the solvent until reaction is complete, typically for between one to 30 hours. The reaction mixture is then cooled and filtered to remove insoluble solids. The solvent is removed under reduced pressure and the crude product is purified by column chromatography or by recrystallisation.

In process (b), a diamine of formula (IIIa) or (IIIb) is treated at a suitable temperature with an excess of an appropriate ortho ester such as triethylorthoformate, triethylorthoacetate, triethylorthopropionate, triethylorthobutanoate, tripropylorthoformate, tributylorthoformate and triisopropylorthoformate, optionally in the presence of a suitable solvent such as an alcohol, until reaction is complete. The temperature is typically up to the reflux temperature of the reaction mixture, and reaction times are generally from 30 minutes to overnight. In one embodiment, the orthoester is triethylorthoformate with ethanol as an optional solvent.

Alternatively in process (b), a diamine of formula (IIIa) or (IIIb) is treated with an alpha-halo-substituted carboxylic acid or anhydride such as trifluoroacetic acid, difluoroacetic acid, fluoroacetic acid, trifluoroacetic anhydride and difluoroacetic anhydride at a suitable temperature between ambient temperature and the reflux temperature of the reaction mixture or in a microwave oven. The process is continued for a suitable period of time, typically for between 0.5 to 5 hours, or 0.1-10 minutes in a microwave oven. After removal of the carboxylic acid or anhydride, treatment with a suitable aqueous base, for example, with 1% or 10% aqueous sodium hydroxide solution, then yields the compound of formula (I). The treatment with base is carried out for a suitable time at a suitable temperature, for example, for about 10 minutes to 4 hours at a temperature between ambient temperature and the reflux temperature of the reaction mixture.

Other methods for the conversion of a diamine of formula (IIIa) or (IIIb) into a compound of formula (Ia) or (Ib) are described in the literature and will be readily known to the person skilled in the art.

The present invention includes compounds of formula (Ia) or (Ib) in the form of salts, in particular acid addition salts. Suitable salts include those formed with both organic and inorganic acids. Such acid addition salts will normally be pharmaceutically acceptable although salts of non-pharmaceutically acceptable acids may be of utility in the preparation and purification of the compound in question. Thus, preferred salts include those formed from hydrochloric, hydrobromic, sulphuric, phosphoric, citric, tartaric, lactic, pyruvic, acetic, succinic, fumaric, maleic, methanesulphonic and benzenesulphonic acids.

Salts of compounds of formula (Ia) or (Ib) may be formed by reacting the free base, or a salt, enantiomer or racemate thereof, with one or more equivalents of the appropriate acid. The reaction may be carried out in a solvent or medium in which the salt is insoluble or in a solvent in which the salt is soluble, for example, water, dioxan, ethanol, tetrahydrofuran or diethyl ether, or a mixture of solvents, which may be removed in vacuo or by freeze drying. The reaction may also be a metathetical process or it may be carried out on an ion exchange resin.

Compounds of formulae (IIa) or (IIb) and compounds of formula (IIIa) or (IIIb) are either known in the literature or may be prepared using known methods that will be readily apparent to the man skilled in the art.

The compounds of the invention and intermediates thereto may be isolated from their reaction mixtures and, if necessary further purified, by using standard techniques.

The compounds of formula (Ia) or (Ib) may exist in enantiomeric forms. Therefore, all enantiomers, diastereomers, racemates and mixtures thereof are included within the scope of the invention. The various optical isomers may be isolated by separation of a racemic mixture of the compounds using conventional techniques, for example, fractional crystallisation, or HPLC. Alternatively, the various optical isomers may be prepared directly using optically active starting materials.

Intermediate compounds may also exist in enantiomeric forms and may be used as purified enantiomers, diastereomers, racemates or mixtures.

The compounds of formula (Ia) or (Ib), and their pharmaceutically acceptable salts are useful because they possess pharmacological activity as inhibitors of the enzyme MPO.

The compounds of formulae (Ia) and (Ib) and their pharmaceutically acceptable salts are indicated for use in the treatment or prophylaxis of diseases or conditions in which modulation of the activity of the enzyme myeloperoxidase (MPO) is desirable. In particular, linkage of MPO activity to disease has been implicated in neuroinflammatory diseases. Therefore the compounds of the present invention are particularly indicated for use in the treatment of neuroinflammatory conditions or disorders in mammals including man. Such conditions or disorders will be readily apparent to the man skilled in the art.

Conditions or disorders that may be specifically mentioned include multiple sclerosis, Alzheimer's disease, Parkinson's disease, amyotrophic lateral sclerosis and stroke, as well as other inflammatory diseases or conditions such as asthma, chronic obstructive pulmonary disease, cystic fibrosis, idiopathic pulmonary fibrosis, acute respiratory distress syndrome, sinusitis, rhinitis, psoriasis, dermatitis, uveitis, gingivitis, atherosclerosis, inflammatory bowel disease, renal glomerular damage, liver fibrosis, sepsis, proctitis, rheumatoid arthritis, and inflammation associated with reperfusion injury, spinal cord injury and tissue damage/scarring/adhesion/rejection. Lung cancer has also been suggested to be associated with high MPO levels. The compounds are also expected to be useful in the treatment of pain.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

For the above mentioned therapeutic indications, the dosage administered will, of course, vary with the compound employed, the mode of administration and the treatment desired. However, in general, satisfactory results are obtained when the compounds are administered at a dosage of the solid form of between 1 mg and 2000 mg per day.

The compounds of formulae (Ia) or (Ib), and pharmaceutically acceptable derivatives thereof, may be used on their own, or in the form of appropriate pharmaceutical compositions in which the compound or derivative is in admixture with a pharmaceutically acceptable adjuvant, diluent or carrier. Thus, another aspect of the invention concerns a pharmaceutical composition comprising a novel compound of formula (Ia) or (Ib), or a pharmaceutically acceptable salt thereof, in admixture with a pharmaceutically acceptable adjuvant, diluent or carrier. Administration may be by, but is not limited to, enteral (including oral, sublingual or rectal), intranasal, inhalation, intravenous, topical or other parenteral routes. Conventional procedures for the selection and preparation of suitable pharmaceutical formulations are described in, for example, “Pharmaceuticals—The Science of Dosage Form Designs”, M. E. Aulton, Churchill Livingstone, 1988. The pharmaceutical composition preferably comprises less than 80% and more preferably less than 50% of a compound of formulae (Ia) or (Ib), or a pharmaceutically acceptable salt thereof.

There is also provided a process for the preparation of such a pharmaceutical composition which comprises mixing the ingredients.

The invention is illustrated, but in no way limited, by the following example:

¹H and ¹³C NMR spectra were recorded either on a 300 MHz Bruker DPX instrument or on a Varian Unity 400 MHz spectrometer at 25° C. The following reference signals were used: the middle line of DMSO-d₆ δ39.5 (¹³C); DMSO-d₆ δ2.49 (¹H). All mass spectra were recorded on a Waters LCMS (2790) instrument. Thin layer chromatography (TLC) was performed on Merck TLC aluminium sheets silica gel 60 F₂₅₄ pre-coated sheets (layer thickness 0.2 mm). Merck Silica gel 60 (0.063-0.200 mm) was used for column chromatography. HPLC analysis were performed on a Agilent 1100 series. Column; Waters X-Terra, C8, 3.5 μm, 4.6×100 mm. Preparative liquid chromatography was performed on a Gilson Auto purification system, gradient pump with a Gynkotek UVD 170S UV-vis detector. Column; Kromasil, C8, 10 μm, 20×250 mm. The microwave oven used is a Smith Creator, Personal Chemistry.

### EXAMPLE 1

### 3-Isobutyl-2-thioxo-8-trifluoromethyl-1,2,3,7-tetrahydro-purin-6-one

5,6-Diamino-1-isobutyl-2-thioxo-2,3-dihydro-1H-pyrimidin-4-one (0.15 g, 0.70 mmol) was suspended in trifluoroacetic acid (3.0 mL) and this solution was heated at 100° C. for 1.5 min in a microwave oven. Excess trifluoroacetic acid was evaporated off under reduced pressure. 0.2M Sodium hydroxide (3.0 mL) was added to the orange solid and the resulting solution was heated at 100° C. for 1.5 minutes in a microwave oven. The pH of the solution was adjusted to pH 6 with dilute hydrochloric acid. The resulting slurry was stirred for 10 min at ambient temperature, then the precipitate was collected by filtration and washed with water. Yield: (0.60 g, 29%).

¹H NMR (400 MHz, DMSO-D6) δ 12.51 (s, 1H), 4.28 (d, J=7.33 Hz, 2H), 2.47 (m, 1H), 0.89 (s, 6H);

¹³C NMR (101 MHz, DMSO-D6) δ 174.51, 152.87, 148.86, 138.47, 118.27, 113.77, 54.22, 26.06, 19.69 (s, 2C);

MS (LC-MS)m/z 291 (M−1).

### Screens

Methods for the determination of MPO inhibitory activity are disclosed in co-pending patent application WO 02/090575. The pharmacological activity of compounds according to the invention was tested in the following screen:

Assay buffer: 20 mM sodium/potassium phosphate buffer pH 6.5 containing 10 mM taurine and 100 mM NaCl.

Developing reagent: 2 mM 3,3′,5,5′-tetramethylbenzidine (TMB), 200 μM KI, 200 mM acetate buffer pH 5.4 with 20% DMF.

To 10 μl of diluted compounds in assay buffer, 40 μl of human MPO (final concentration 2.5 nM) was added for 10 minutes at room temperature. Then 50 μl of H₂O₂ (final concentration 100 μM), or assay buffer alone as a control, were added for 10 minutes at room temperature. The reaction was stopped by adding 10 μl 0.2 mg/ml of catalase (final concentration 18 μg/ml) for 5 minutes before 100 μl of TMB developing reagent was added (2 mM TMB in 200 mM acetate buffer pH 5.4 containing 20% dimethylformamide (DMF) and 200 μM KI). Plates were mixed and the amount of oxidised 3,3′,5,5′-tetramethylbenzidine formed was then measured after about 5 minutes using absorbance spectroscopy at about 650 nM. IC₅₀ values were then determined using standard procedures.

When tested in the above screen, the compound of Example 1 gave an IC₅₀ value of less than 60 μM, indicating that it is expected to show useful therapeutic activity.

1-10. (canceled)

11. A compound of formula (Ia) or (Ib) 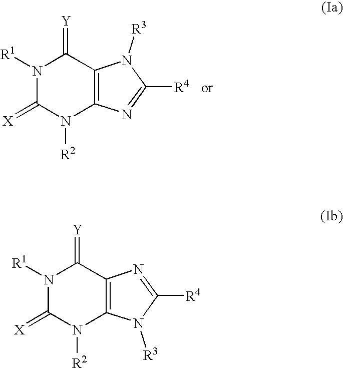
wherein:
one of X and Y represents S, and the other represents O or S;
R¹ represents hydrogen or C1 to 6 alkyl;
R² represents hydrogen or C1 to 6 alkyl; said alkyl group being optionally substituted by:
i) a saturated or partially unsaturated 3- to 7-membered ring optionally incorporating one or two heteroatoms selected independently from O, N and S, and optionally incorporating a carbonyl group; said ring being optionally substituted by one or more substituents selected from halogen, hydroxy, C1 to 6 alkoxy and C1 to 6 alkyl; said alkyl being optionally further substituted by hydroxy or C1 to 6 alkoxy; or
ii) C1 to 6 alkoxy; or
iii) an aromatic ring selected from phenyl, furyl or thienyl; said aromatic ring being optionally further substituted by halogen, C1 to 6 alkyl or C1 to 6 alkoxy;
R³ represents hydrogen or C1 to 6 alkyl;
R⁴ represents halogen, C1 to 6 alkyl substituted by one or more halogen atoms, C1 to 6 alkoxy or C1 to 6 thioalkoxy; said alkoxy or thioalkoxy group being optionally further substituted by halogen or OH;
and pharmaceutically acceptable salts thereof.

12. A compound according to claim 11 wherein X represents S and Y represents O.

13. A compound according to claim 11 wherein R³ represents H.

14. A compound according to claim 11 wherein R² represents optionally substituted C1 to 6 alkyl.

15. A compound of formula (Ia) or (Ib), according to claim 11, or a pharmaceutically acceptable salt thereof, for use as a medicament.

16. A pharmaceutical composition comprising a compound of formula (Ia) or (Ib) according to claim 11, or a pharmaceutically acceptable salt thereof, optionally in admixture with a pharmaceutically acceptable adjuvant, diluent or carrier.

17. A method of treating, or reducing the risk of, diseases or conditions in which inhibition of the enzyme MPO is beneficial which comprises administering to a person suffering from or at risk of, said disease or condition, a therapeutically effective amount of a compound of formula (Ia) or (Ib), as defined in claim 11 or a pharmaceutically acceptable salt thereof.

18. The use of a compound of formula (Ia) or (Ib) as defined in claim 11 or a pharmaceutically acceptable salt thereof, in the manufacture of a medicament, for the treatment or prophylaxis of diseases or conditions in which inhibition of the enzyme MPO is beneficial.

19. The use of a compound of formula (Ia) or (Ib) as defined in claim 11 or a pharmaceutically acceptable salt thereof, in the manufacture of a medicament, for the treatment or prophylaxis of neuroinflammatory disorders.

20. A process for the preparation of a compound of formula (Ia) or (Ib), as defined in claim 11 or a pharmaceutically acceptable salt, enantiomer, diastereomer or racemate thereof, wherein the process comprises:
(a) reaction of a compound of formula (IIa) or (IIb) 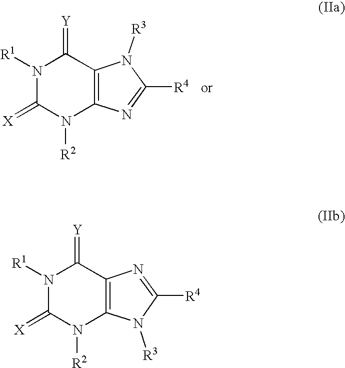
wherein R¹, R², R³ and R⁴ are as defined in formula (Ia) or (Ib), X represents O or S and Y represents O;
with a sulphurising compound such as Lawesson's reagent or phosphorus pentasulphide;
to give a corresponding compound wherein Y represents S; or
(b) reaction of a diamine of formula (IIIa) or (IIIb) 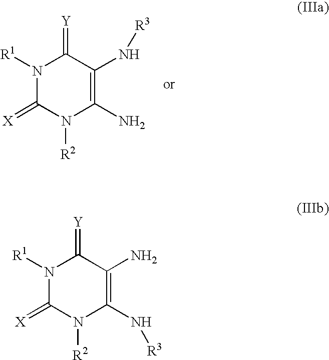
wherein R¹, R², R³, X and Y are as defined in formula (Ia) or (Ib);
with a trialkylorthoester or with an alpha-halo-substituted carboxylic acid or anhydride;
and where necessary converting the resultant compound of formula (Ia) or (Ib), or another salt thereof, into a pharmaceutically acceptable salt thereof; or converting the resultant compound of formula (Ia) or (Ib) into a further compound of formula (Ia) or (Ib); and where desired converting the resultant compound of formula (Ia) or (Ib) into an optical isomer thereof.
